(19) United States
(12) Patent Application Publication
(10) Pub. No.: US 2003/0040477 A1
(43) Pub. Date: Feb. 27, 2003

(54) Methods and compositions for modulating t cell activation and uses thereof

(76) Inventors: Anthony Montgomery, San Diego, CA (US); Larissa Balaian, San Diego, CA (US)

Correspondence Address: Peng Chen, Morrison & Foerster, Suite 500, 3811 Valley Centre Drive, San Diego, CA, 92130-2332, US

(21) Appl. No.: 10/130,087

(22) PCT Filed: Oct. 2, 2001
(86) PCT No.: PCT/US01/30864

(51) Int. Cl. A61K 38/17; A61K 48/00

(52) U.S. Cl. 514/12; 514/44

(57) ABSTRACT

This invention relates generally to the field of immunology or neuroimmunology. In particular, the invention provides a method for reducing or inhibiting T cell activation, which method comprises administering an effective amount of an antagonist of NCAM L1 to a mammal, wherein reduction or inhibition of T cell activation is desirable, thereby reducing or inhibiting T cell activation in said mammal. Combinations and combinatorial methods for modulating T cell activation are further provided. The invention also provides a method for potentiating T cell activation, which method comprises administering an effective amount of a multimerized neural cell adhesion molecule L1 (NCAM L1), or a functional derivative or fragment thereof, or a nucleic acid encoding said L1 or func tional derivative or fragment thereof, or an agent that enhances production and/or costimulatory function of said L1 to a mammal, wherein T cell activation is desirable, thereby potentiating T cell activation in said mammal.

### TECHNICAL FIELD

This invention relates generally to the field of immunology or neuroimmunology. In particular, the invention provides a method for reducing or inhibiting T cell activation, which method comprises administering an effective amount of an antagonist of NCAM L1 to a mammal, wherein reduction or inhibition of T cell activation is desirable, thereby reducing or inhibiting T cell activation in said mammal. Combinations and combinatorial methods for modulating T cell activation are further provided. The invention also provides a method for potentiating T cell activation, which method comprises administering an effective amount of a multimerized neural cell adhesion molecule L1 (NCAM L1), or a functional derivative or fragment thereof, or a nucleic acid encoding said L1 or functional derivative or fragment thereof, or an agent that enhances production and/or costimulatory function of said L1 to a mammal, wherein T cell activation is desirable, thereby potentiating T cell activation in said mammal.

### BACKGROUND ART

Current paradigms of T-cell activation are based on the premise that optimal activation requires two signals; the first being provided by occupancy of the T-cell receptor (TCR) by MHC/antigen complex, the second being provided by one or more costimulatory ligands on the surface of the APC (1). An array of molecules on the surface of the APC can function as costimulatory ligands including members of the immunoglobulin superfamily (IgSF) such as B7-1, B7-2 and ICAM-1 (1).

Previous studies identified L1 as a neuronal CAM that also belongs to the IgSF (2). To date, L1 function has almost exclusively been linked to neurological processes, including axonal guidance (3,4). While such L1-mediated processes have primarily been attributed to homophilic L1-L1 ligation (5), this CAM can also interact with multiple heterophilic ligands including axonin 1/TAG 1, chondroitin sulfate proteoglycans, laminin and certain integrins (6,7). L1 has also been shown to support cis-interactions with the heat stable antigen CD24 (8) and the tetraspan molecule CD9 (9).

Despite its neuronal designation, L1 expression has recently been described on cells of both lymphoid and myelomonocytic origin (10, 11). Specifically, L1 can be detected on freshly isolated peripheral blood monocytes and on functionally mature monocyte-derived dendritic cells (DC) and on follicular DC in situ (11). Further constitutive expression is evident on a subset of B-cells and has been described on CD4+ T-cells (10,11). Despite these findings, little is known of the function of L1 in the immune system. One recent study has shown that L1 is important for the maintenance of lymph node architecture (12). Using a variety of experimental approaches, including one-way MLR and mitogen-activation assays, we demonstrate here that L1 can function as a costimulatory molecule in T-cell activation. In this capacity, L1 contributes to the initiation of human immune responses in normal and disease processes including those involving the nervous system.

Accordingly, it is an object of the present invention to provide methods for modulating T cell activation using NCAM L1 as the modulating target. It is another objective of the present invention to provide combinations and combinatorial methods for modulating T cell activation.

### DISCLOSURE OF THE INVENTION

This invention relates generally to the field of immunology or neuroimmunology. In one aspect, the invention provides a method for potentiating T cell activation, which method comprises administering an effective amount of a multimerized neural cell adhesion molecule L1 (NCAM L1), or a functional derivative or fragment thereof, or a nucleic acid encoding said L1 or functional derivative or fragment thereof, or an agent that enhances production and/or costimulatory function of said L1 to a mammal, wherein T cell activation is desirable, thereby potentiating T cell activation in said mammal.

Any multimerized, e.g., dimerized, NCAM L1, or a functional derivative or fragment thereof, that can function as a stimulatory molecule in T cell activation, and any nucleic acids encoding such NCAM L1, or functional derivative or fragment thereof, can be used in the present methods. Preferably, the NCAM L1, or a functional derivative or fragment thereof, is capable of L1-L1 homophilic interaction, e.g. mediating a L1-L1 ligation between an antigen presentation cell (APC) and a T cell. Also preferably, the NCAM L1, or a functional derivative or fragment thereof, supports an interaction with an integrin involved in T cell activation, e.g., supporting a trans or cis interaction with the integrn α5β1 or αvβ3. Further preferably, the NCAM L1, or a functional derivative or fragment thereof, supports an interaction with a ligand involved in costimulation, e.g., supporting a cis-type interaction with CD9 and/or CD24.

Any agents that enhances production and/or costimulatory function of NCAM L1 can be used in the present methods. Preferably, the agents used therein enhance L1-L1 homophilic interaction between two NCAM L1, or a functional derivative or fragment thereof, or interaction between a NCAM L1, or a functional derivative or fragment thereof, and an integrin involved in T cell activation, or interaction between a NCAM L1, or a functional derivative or fragment thereof, and a ligand involved in costimulation. One exemplary agent is the anti-NCAM L1 monoclonal antibody 557.B6 (Appel et al., J. Neurobiol., 28(3):297-312 (1995)).

NCAM L1, or a functional derivative or fragment thereof, from any mammalian origins can be used. Preferably, when the mammal to be treated is a human, the NCAM L1, or a functional derivative or fragment thereof, of human origin is used.

The present methods can be used to activate CD4⁺ T cells, CD8⁺ T cells or both. The present methods can be used to treat, either prophylactically or therapeutically, mammals with diseases or disorders associated with deficient T cell activation. Examples of such diseases or disorders include, but are not limited to, tumors, cancers and infections. Mammals, preferably humans, with tumors, cancers or infections are treated with the present methods.

In another aspect, the invention is directed to a combination, which combination comprises: a) an effective amount of multimerized NCAM L1 or a functional derivative or fragment thereof or a nucleic acid encoding said L1 or functional derivative or fragment thereof or an agent that enhances production and/or costimulatory function of said L1; and b) an effective amount of another costimulatory molecule. Preferably, the combination is in the form of a pharmaceutical composition. Additionally, the invention is directed to a method for potentiating T cell activation, which method comprises administering an effective amount of multimerized NCAM L1 or a functional derivative or fragment thereof, or a nucleic acid encoding said L1 or functional derivative or fragment thereof or an agent that enhances production and/or costimulatory function of said L1 and an effective amount of another costimulatory molecule to a mammal, wherein T cell activation is desirable, thereby potentiating T cell activation in said mammal. Any costimulatory molecules can be used in the above combinations and methods. Preferably, the costimulatory molecules used are CD28, OX40, 4-1BB or ICOS. Also preferably, the costimulatory molecule is derived from an antigen presenting cell (APC), e.g., LFA-1, LFA-3, ICAM-1, ICAM-2, ICAM-3, CD 40 or B7.

In still another aspect, the invention also provides a method for reducing or inhibiting T cell activation, which method comprises administering an effective amount of an antagonist of NCAM L1 to a mammal, wherein reduction or inhibition of T cell activation is desirable, thereby reducing or inhibiting T cell activation in said mammal.

Any antagonists that reduce or inhibit production and/or costimulatory function of NCAM L1 can be used in the present methods. The antagonists can be NCAM L1 anti-sense oligonucleotides, anti-NCAM L1 antibodies, especially monoclonal antibodies such as mAb 5G3, soluble NCAM L1, or derivatives or fragments thereof. The antagonists can reduce or inhibit L1-L1 homophilic interaction, e.g., L1-L1 ligation between an antigen presentation cell and a T cell. The antagonists can reduce or inhibit a L1-L1 ligation without simultaneously causing NCAM L1 clustering and signaling. The antagonists can reduce or inhibit NCAM L1's interaction with an integrin involved in T cell activation, e.g., NCAM L1's trans or cis interaction with the integrin α5β1 or αvβ3, or reduce or inhibit NCAM L1's interaction with a ligand involved in costimulation, e.g., NCAM L1's interaction with CD9 and/or CD24. Preferably, the NCAM L1 antagonists used in the methods or combinations are protein, polypeptide or peptide antagonists. Also preferably, the NCAM L1 antagonists used in the methods or combinations are small molecule antagonists, e.g., ethanol (Bearer et al., J. Biol. Chem., 274(19):13264-13270 (1999)).

The present methods can be used to reduce or inhibit activation of CD4⁺ T cells, CD8⁺ T cells or both.

The present methods can be used to treat, either prophylactically or therapeutically, mammals with diseases or disorders associated with undesirable T cell activation. Examples of such diseases or disorders include, but are not limited to, autoimmunity, graft rejection and neuroimmunological disorders. Mammals, preferably humans, with autoimmunity, graft rejection and neuroimmunological disorders are treated with the present methods.

In yet another aspect, the invention is directed to a combination, which combination comprises: a) an effective amount of an antagonist of NCAM L1; and b) an effective amount of another costimulatory inhibitory molecule. Preferably, the combination is in the form of a pharmaceutical composition. Additionally, the invention is directed to a method for reducing or inhibiting T cell activation, which method comprises administering an effective amount of an antagonist of NCAM L1 and an effective amount of another costimulatory inhibitory molecule to a mammal, wherein T cell reduction or inhibition is desirable, thereby reducing or inhibiting T cell activation in said mammal. Any costimulatory inhibitory molecules can be used in the above combinations and methods. Preferably, the costimulatory inhibitory molecules used is T-lymphocyte-associated antigen 4 (CTLA-4).

### BRIEF DESCRIPTION OF THE DRAWINGS

FIG. 1. L1 is expressed by cord blood-derived DC and contributes to allogenic MLR. (A), CD34+-cells were enriched from normal cord blood and expanded for 21 days prior to staining for L1-expression. Enriched CD34+-cells were also stained for L1 prior to culture (Day 0). The cells were stained with mAb 5G3 directly conjugated to FITC. (B), Cord blood derived DC (DC+) were cocultured with PBMC from a different donor in an one way MLR. Cells were cultured in the presence or absence of anti-L1 mAb 5G3 or with control antibody UPC10. CD34+-negative cord blood cells cultured under identical conditions as the CD34+ enriched fraction were also tested as stimulators (non-DC). Cultures were pulsed with [3H]-thymidine during the last 18 hours of a three day coculture. Treatments were performed in triplicate. Error bars are ±1SE.

FIG. 2. Anti-L1 antibody 5G3 and soluble L1 inhibit autologous T-cell responses to mitogen. (A) PBMC were treated with PHA (10 mg/ml) in the absence or presence of mAb 5G3, or in the presence of control antibody UPC10. Further PBMC were cultured in the presence of soluble recombinant L1-ECD (sL1; 100 mg/ml). (B) PBMC were treated with range of PHA concentrations in the absence or presence of mAb 5G3, or in the presence of control antibody UPC10. Cultures were pulsed with [3H]-thymidine during the last 18 hours of a three day coculture. (B, inset) Some cells were treated with PHA and pulsed with [3H]-thymidine for 18 hours only. (C) Enriched CD4+ or CD8+ T-cell subsets were treated with PHA in the absence or presence of mAb 5G3, or in the presence of control antibody UPC10. Cultures were pulsed with [3H]-thymidine during the last 18 hours of a three day coculture. Treatments were performed in triplicate. Error bars are ±1SE.

FIG. 3. Purified immobilized L1 potentiates T-cell proliferation in response to CD3 ligation. Wells of a 96-well plate were pretreated with anti-CD3 antibody (OKT3: 25 U/ml), with purified L1-ectodomain (40 mg/ml) or with a combination of both L1 and the antibody. After washing the wells, PBMCs were added to the precoated wells or to untreated wells for 72 hours. PBMC were cultured in the absence or presence of mAb 5G3, or in the presence of control antibody UPC10. Cultures were pulsed with [3H]-thymidine during the last 18 hours of a three day coculture. Treatments were performed in triplicate. Error bars are ±1SE.

FIG. 4. Transfection and de novo expression of L1 enhances MLR. (A & B) Irradiated wildtype (WT cells) or L1-transfected J558L myeloma cells (L1+ cells) were cocultured with PBMC (A) or enriched CD4+ or CD8+ T-cell subsets (B) in an one way MLR L1+J558L myeloma cells were co-cultured in the presence or absence of anti-L1 mAb 5G3 (80 mg/ml) or in the presence of control mAb UPC10 (80 mg/ml). (A, inset) Further co-cultures of L1+J558L myeloma cells and PBMC were incubated with mAb 5G3 at concentrations varying from 20-160 mg/ml (inset). Cultures were pulsed with [3H]-thymidine during the last 18 hours of a three day coculture. Treatments were performed in triplicate. Error bars are ±1SE.

FIG. 5. Inhibitory antibody 5G3 blocks L1-L1 mediated adhesion by L1 transfected myeloma cells. Wildtype (WT) or L1-transfected (L1+) myeloma cells were allowed to adhere to immobilized recombinant L1 in the presence of absence of control mAb (UPC10) or anti-L1 mAb (5G3). Adherent cells were counted per unit area with a 40× high powered objective. Experimental treatments were performed in triplicate with four areas counted per well. Error bars represent ±1 SD.

### MODES OF CARRYING OUT THE INVENTION

A. Definitions

Unless defined otherwise, all technical and scientific terms used herein have the same meaning as is commonly understood by one of ordinary skill in the art to which this invention belongs. All patents, applications, published applications and other publications and sequences from GenBank and other data bases referred to herein are incorporated by reference in their entirety.

As used herein, “T cell activation” refers to cellular activation of resting T cell manifesting a variety of responses that include T cell proliferation, cytokine secretion and/or effector function. T cell activation may be induced by stimulation of the T cell receptor (TCR) with antigen/MHC complex. Alternatively, T cell activation may be induced by specified lectins, e.g., phytohemagglutinin, or monoclonal antibody(ies) to TCR.

As used herein, “costimulatory molecule” refers to molecules that modulate the outcome of prior engagement of the TCR augmenting T cell activation events including T-cell proliferation and effector function. Signals provide by costimulatory molecules are not antigen specific nor MHC-restricted, and by themselves, Le., in the absence of TCR engagement, are unable to induce a significant response in T cells. Engagement of the TCR in the absence of costimulatory molecules result in no immune response or hyporesponsiveness.

As used herein, “neural cell adhesion molecule L1 (NCAM L1)” refers to a neural cell adhesion molecule that belongs to the IgSF superfamily and can function as a costimulatory molecule in T cell activation. NCAM L1 can exerts its costimulatory function through L1-L1 homophilic interaction, e.g., mediating a L1-L1 ligation between APCs and T cells or through interaction with an integrin involved in T cell activation, e.g., the integrin α5β1 or αvβ3, or through interaction with a ligand involved in costimulation, e.g., CD9 and/or CD24. Preferably, NCAM L1 has 6 immunoglobulin like domains, and has 5 fibronectin type III like domains, and is a membrane-penetrating type glycoprotein expected to penetrate the membrane at a region having sufficient number, e.g., 23, hydrophobic amino acid residues starting with an amino acid with a small side chain, e.g., glycine (EP 0,572,664 A1; and Moos et al., Nature, 334: 701-703 (1988)). It is intended that NCAM L1 includes those variants with conservative amino acid substitutions that do not substantially alter its costimulatory activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. Watson et al. Molecular Biology of the Gene, 4th Edition, 1987, The Bejacmin/Cummings Pub. co., p.224).

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

It is understood that equivalent stringencies may be achieved using alternative buffers, salts and temperatures.

As used herein, a “functional derivative or fragment of NCAM L1” refers to a derivative or fragment of NCAM L1 that still substantially retains its function as a costimulatory molecule. Normally, the derivative or fragment retains at least 1%, 10%, 20%, 30%, 40%, 50% of its costimulatory activity. Preferably, the derivative or fragment retains at least 60%, 70%, 80%, 90%, 95%, 99% and 100% of its costimulatory activity. Functional derivative or fragment of NCAM L1 also encompasses peptide or polypeptide derivative or fragment of NCAM L1 that substantially retains its function as a costimulatory molecule.

As used herein, an “agent that enhances production of NCAM L1” refers to a substance that increases transcription and or translation of a NCAM L1 gene, or a substance that increases post-translational modification and/or cellular trafficking of a NCAM L1 precursor, or a substance that prolongs half-life of a NCAM L1 protein.

As used herein, an “agent that enhances costimulatory function of NCAM L1” refers to a substance that increases potency of NCAM L1's costimulatory activity, or a substance that increases sensitivity of a NCAM L1's natural ligand in a costimulatory signally pathway, or a substance that decreases potency of a NCAM L1's antagonist.

As used herein, “integrins” refers to a family of cell membrane glycoproteins that are heterodimers composed of α and β-chain subunits. They serve as glycoprotein receptors involved in cell-cell or cell-substrate adhesion, e.g., the mediation of adhesion of neutrophils to endothelial cells, or to extracellular matrix such as collagen.

As used herein, “neoplasm (neoplasia)” refers to abnormal new growth, and thus means the same as tumor, which may be benign or malignant. Unlike hyperplasia, neoplastic proliferation persists even in the absence of the original stimulus.

As used herein, “cancer” refers to a general term for diseases caused by any type of malignant tumor.

As used herein, an “antagonist of NCAM L1 (or NCAM L1 antagonist)” refers to a substance that decreases production and/or costimulatory function of NCAM L1. Such an antagonist can decrease production of NCAM L1 by decreasing transcription and or translation of a NCAM L1 gene, or by decreasing post-translational modification and/or cellular trafficking of a NCAM L1 precursor, or by shortening half-life of a NCAM L1 protein. Such an antagonist can decrease costimulatory function of NCAM L1 by decreasing potency of NCAM L1's costimulatory activity, or by decreasing sensitivity of a NCAM L1's natural ligand in a costimulatory signally pathway, or by increasing potency of a NCAM L1's antagonist. NCAM L1 antagonist can be any type of substances, including protein, polypeptide, peptide, or small molecule antagonist.

As used herein, “antibody” includes antibody fragments, such as Fab fragments, which are composed of a light chain and the variable region of a heavy chain

As used herein, a “combination” refers to any association between two or among more items.

As used herein, a “composition” refers to a any mixture of two or more products or compounds. It may be a solution, a suspension, liquid, powder, a paste, aqueous, non-aqueous or any combination thereof.

As used herein, “antisense polynucleotides” refer to synthetic sequences of nucleotide bases complementary to mRNA or the sense strand of double stranded DNA. Admixture of sense and antisense polynucleotides under appropriate conditions leads to the binding of the two molecules, or hybridization. When these polynucleotides bind to (hybridize with) mRNA, inhibition of protein synthesis (translation) occurs. When these polynucleotides bind to double stranded DNA, inhibition of RNA synthesis (transcription) occurs. The resulting inhibition of translation and/or transcription leads to an inhibition of the synthesis of the protein encoded by the sense strand.

As used herein, an “NCAM L1 antisense oligonucleotide” refers to any oligomer that prevents production or expression of NCAM L1 polypeptide. The size of such an oligomer can be any length that is effective for this purpose. In general, the antisense oligomer is prepared in accordance with the nucleotide sequence of a portion of the transcript of NCAM L1 that includes the translation initiation codon and contains a sufficient number of complementary nucleotides to block translation.

As used herein, an “autoimmunity” refers to specific humoral or cell-mediated immune response to the body's own tissues.

For clarity of disclosure, and not by way of limitation, the detailed description of the invention is divided into the subsections that follow.

B. Methods for Potentiating T Cell Activation

In one aspect, the invention provides a method for potentiating T cell activation, which method comprises administering an effective amount of a multimerized neural cell adhesion molecule L1 (NCAM L1), or a functional derivative or fragment thereof, or a nucleic acid encoding said L1 or functional derivative or fragment thereof; or an agent that enhances production and/or costimulatory function of said L1 to a mammal, wherein T cell activation is desirable, thereby potentiating T cell activation in said mammal.

Any multimerized, e.g., dimerized, NCAM L1, or a functional derivative or fragment thereof; that can function as a stimulatory molecule in T cell activation, and any nucleic acids encoding such NCAM L1, or functional derivative or fragment thereof; can be used in the present methods.

For example, NCAM L1 proteins with the following GenBank accession numbers can be used: T30532 (Fugu rubripes); T30581 (zebra fish); S36126 (rat); A43425 (chicken); S05479 (mouse); A41060 (human); NP_{—}032504 (Mus musculus); NP_{—}006605 (close homologue of L1 sapiens); NP_{—}000416 (Homo sapiens); AAF22153 (Mus musculus); CAB57301 (Mus musculus); P32004 (HUMAN); Q05695 (RAT); P11627 (MOUSE); AAD28610 (Cercopithecus aethiops); CAB37831 (Homo sapiens); AAC51746 (Homo sapiens); AAC15580 (Fugu rubripes); AAC14352 (Homo sapiens); CAA96469 (Fugu rubripes); CAA82564 (Homo sapiens); CAA41576 (Homo sapiens); 1411301A; CAA42508 (Homo sapiens); CAA41860 (Rattus norvegicus); AAA99159 (Carassius auratus); CAA61491 (Danio rerio); CAA61490 (Danio rerio); AAA59476 (Homo sapiens); AAA36353 (Homo sapiens). In addition, any proteins derived from, or are portion of, the above NCAM L1 proteins that still substantially retain their costimulatory activities can be used. Preferably, such NCAM L1 derivatives or fragments can be recognized by antibodies that specifically recognize the NCAM L1 proteins from which the derivatives or fragments originate.

Similarly, nucleic acids encoding NCAM L1 proteins with the following GenBank accession numbers can be used: AC005775 (Homo sapiens); AC 004690 (Homo sapiens); M28231 (Drosophila melanogaster neuroglian precursor); AH006326 (Drosophila melanogaster neuroglian (nrg), alternative splice products); AF050085 (Drosophila melanogaster neuroglian (nrg) gene; AF172277 (Homo sapiens); AF133093 (Mus musculus); AJ239325 (Homo sapiens); AL021940 (Homo sapiens); AF129167 (Chlorocebus aethiops); AJ011930 (Homo sapiens); U52112 (Homo sapiens); M97161 (Rattus norvegicus); AC005626 (Homo sapiens); AF026198 (Fugu rubripes); M77640 (Homo sapiens); U55211 (Carassius auratus); M74387 (Human). In addition, any nucleic acids derived from, or are portion of, the above nucleic acids encoding NCAM L1 that still substantially retain their costimulatory activities can be used. Preferably, such NCAM L1 nucleic acid derivatives or fragments can hybridize under low, middle or high stringency with the NCAM L1 nucleic acids from which the derivatives or fragments originate.

Preferably, the NCAM L1, or a functional derivative or fragment thereof, is capable of L1-L1 homophilic interaction, e.g., mediating a L1-L1 ligation between an antigen presentation cell (APC) and a T cell. Also preferably, the NCAM L1, or a functional derivative or fragment thereof, supports an interaction with an integrin involved in T cell activation, e.g., supporting a trans or cis interaction with the integrin α5β1 (Ruppert et al., J. Cell Biol., 131:1881-1891 (1995)), or integrin α5β3 (Sturmhofel et al., J. Immunol., 154(5):2104-11 (1995); and Poul et al., Mol. Immunol., 32(2):101-16 (1995)), or CD11c, a β2 integrin (Meunier, et al., J. Invest. Dermatol., 103(6):775-9 (1994)), or VLA integrin family (Dang, et al., J. Exp. Med., 172(2):649-52 (1990)). Further preferably, the NCAM L1, or a functional derivative or fragment thereof, supports an interaction with a ligand involved in costimulation, e.g., supporting a cis-type interaction with CD9 and/or CD24 (Liu et al., J. Exp. Med., 175::437-445 (1992); and Lagaudriere-Gesbert et al., Cell. Immunol., 182:105-112 (1997)).

The NCAM L1, or functional derivative or fragment thereof, or the nucleic acid encoding the NCAM L1, or functional derivative or fragment thereof, can be administered to the mammal by any methods know in the art For example, the NCAM L1, or functional derivative or fragment thereof, or the nucleic acid encoding the NCAM L1, or functional derivative or fragment thereof, can be administered directly to the mammal. Alternatively, the NCAM L1, or functional derivative or fragment thereof, or the nucleic acid encoding the NCAM L1, or functional derivative or fragment thereof, can be delivered into antigen presenting cells, e.g., macrophages and dendritic cells, and the antigen presenting cells containing the NCAM L1 or the nucleic acid are then administered to the mammal.

Any agents that enhances production and/or costimulatory function of NCAM L1 can be used in the present methods. Preferably, the agents used therein enhance L1-L1 homophilic interaction between two NCAM L1, or a functional derivative or fragment thereof, or interaction between a NCAM L1, or a functional derivative or fragment thereof, and an integrin involved in T cell activation, or interaction between a NCAM L1, or a functional derivative or fragment thereof, and a ligand involved in costimulation.

NCAM L1, or a functional derivative or fragment thereof, from any mammalian origins can be used. Preferably, when the mammal to be treated is a human, the NCAM L1, or a functional derivative or fragment thereof, of human origin is used.

The present methods can be used to activate CD4⁺ T cells, CD8⁺ T cells or both. The present methods can be used to treat, either prophylactically or therapeutically, mammals with diseases or disorders associated with deficient T cell activation. Examples of such diseases or disorders include, but are not limited to, tumors, cancers or infections. Examples of tumors or cancers that can be treated with the present methods include breast cancer, Burkitt lymphoma, colon cancer, small cell lung carcinoma, melanoma, multiple endocrine neoplasia (MEN), neurofibromatosis, p53-associated tumor, pancreatic carcinoma, prostate cancer, Ras-associated tumor, retinoblastoma and Von-Hippel Lindau disease (VHL). Preferably, tumors or cancers that originate from immune system and/or nervous system are treated.

Any mammals, such as, mice, rats, rabbits, cats; dogs, pigs, cows, ox, sheep, goats, horses, monkeys and other non-human primates, with tumors, cancers or infections can be treated with the present methods. Preferably, humans with tumors or cancers are treated with the present methods.

C. Methods for Inhibiting T Cell Activation

In another aspect, the invention is directed to a method for reducing or inhibiting T cell activation, which method comprises administering an effective amount of an antagonist of NCAM L1 to a mammal, wherein T cell reduction or inhibition is desirable, thereby reducing or inhibiting T cell activation in said mammal.

Any antagonists that reduce or inhibit production and/or costimulatory function of NCAM L1 can be used in the present methods. Preferably, the antagonists used therein are NCAM L1 anti-sense oligonucleotides, anti-NCAM L1 antibodies, especially monoclonal antibodies such as mAb 5G3 (Balaian et al., Eur. J. Immunol., 30(3):938-43 (2000)), soluble NCAM L1, or derivatives or fragments thereof. Also preferably, the antagonists of NCAM L1 used reduce or inhibit L1-L1 homophilic interaction, e.g., L1-L1 ligation between an antigen presentation cell and a T cell. More preferably, the antagonists reduce or inhibit a L1-L1 ligation without simultaneously causing NCAM L1 clustering and signaling. Still preferably, the antagonists of NCAM L1 used reduce or inhibit NCAM L1's interaction with an integrin involved in T cell activation, e.g., NCAM L1's trans or cis interaction with the integrin α5β1, αvβ3, CD11c, a β2 integrin or VLA integrin family, or reduce or inhibit NCAM L1's interaction with a ligand involved in costimulation, e.g., NCAM L1's interaction with CD9 and/or CD24.

In a preferred embodiment, the NCAM L1 antagonists used in the methods or combinations are protein, polypeptide or peptide antagonists. In another preferred embodiment, the NCAM L1 antagonists used in the methods or combinations are small molecule antagonists, e.g., ethanol (Bearer et al., J. Biol. Chem., 274(19):13264-13270 (1999)).

The present methods can be used to reduce or inhibit activation of CD4⁺ T cells, CD8⁺ T cells or both.

The present methods can be used to treat, either prophylactically or therapeutically, mammals with diseases or disorders associated with undesirable T cell activation. Examples of such diseases or disorders include, but are not limited to, autoimmunity, graft rejection and neuroimmunological disorders. Mammals, preferably humans, with autoimmunity, graft rejection and neuroimmunological disorders are treated with the present methods.

D. Combinations and Combinatorial Treatments

In still another aspect, the invention is directed to a combination, which combination comprises: a) an effective amount of multimerized NCAM L1 or a functional derivative or fragment thereof, or a nucleic acid encoding said L1 or functional derivative or fragment thereof, or an agent that enhances production and/or costimulatory function of said L1; and b) an effective amount of another costimulatory molecule, or an immunostimulant such as an agonist of costimulatory molecules, or certain cytokines, e.g., IL-2. Preferably, the combination is in the form of a pharmaceutical composition. Additionally, the invention is directed to a method for potentiating T cell activation, which method comprises administering an effective amount of multimerized NCAM L1 or a functional derivative or fragment thereof, or a nucleic acid encoding said L1 or functional derivative or fragment thereof, or an agent that enhances production and/or costimulatory function of said L1 and an effective amount of another costimulatory molecule or an immunostimulant to a mammal, wherein T cell activation is desirable, thereby potentiating T cell activation in said mammal. Any costimulatory molecules can be used in the above combinations and methods. Preferably, the costimulatory molecules used are CD28, OX40, 4-1BB or ICOS.

CD28 is the primary positive T cell costimulatory molecule, as defined by the ability to enhance T cell activation in the presence of TCR stimulation that is insufficient for T cell proliferation (see generally Chambers and Allison, Curr. Opin. Cell. Biol., 11(2):203-10 (1999)). CD28 is an immunoglobulin supergene family glycoprotein that is expressed as homodimers on T cells. It binds to ligands B7.1 and B7.2 via the MYPPPY (in the single letter code for amino acids) motif in the immunoglobulin domain. The cytoplasmic tail of CD28 possesses tyrosine-containing motifs postulated to be involved in signal transduction and protein trafficking. In a specific embodiment, CD28 protein with the following GenBank accession numbers can be used in the combination and combinatorial treatment method. NP_{—}006130 (Homo sapiens); B45895 (human); I49584 (mouse); I46689 (rabbit); S24413 (rat); A43523 (mouse); RWHU28 (human); AAF45150 (Mus musculus); BAA92349 (Felis catus); AAF36501 (Marmota monax); NP_{—}037253 (Rattus norvegicus); AAF33794 (Homo sapiens); AAF33793 (Homo sapiens); AAF33792 (Homo sapiens); BAA08641 (Oryctolagus cuniculus); NP_{—}031668 (Mus musculus); NP_{—}008820 (Homo sapiens); NP_{—}005182 (Homo sapiens); Q28071 (Bovin); P42069 (rabbit); P31042 (rat); P31041 (mouse); P31043 (chick); P10747 (human) AD04379 (Ovis arie gi); AAB53574 (Felis catus); CAA63707 (Bos taurus); CAA39003 (Rattus norvegicus); AAA51945 (Homo sapiens); AAA51944 (Homo sapiens); AAA37395 (Mus musculus); AAA37396 (Mus musculus). Similarly, nucleic acids encoding CD28 with the following GenBank accession numbers can be used in the combination and combinatorial treatment method: AB025316 (Felis catus); AF130427 (Marmota monax); AF222343 (Homo sapiens); AF222342 (Homo sapiens); AF222341 (Homo sapiens); D49841 (rabbit); AF092739 (ovis aries); A1528690 (mouse); AI386096 (human); AI327367 (mouse); AI324382 (mouse); AII52205 (mouse); AA940559 (mouse); U57754 (Felis catus); AA17418 (human); AA163825 (mouse); J02988 (human); M34563 (mouse).

In another specific embodiment, OX40 protein with the following GenBank accession numbers can be used in the combination and combinatorial treatment method: 137552 (OX40 homolog-human); JE0351 (rat); 148700 (mouse); S48290 (mouse); S12783 (rat); 1D0AL 1D0AK (Chain L, Human); 1D0AJ (Chain J, Human); 1D0AI (Chain I, Human); 1D0AH (Chain H, Human); 1D0AG (Chain G, Human); 1D0AF (Chain F, Human); 1D0AE Chain E, Human); 1D0AD (Chain D, Human); ID0AC (Chain C, Human); 1D0AB (Chain B, Human); 1D0AA (Chain A, Human); NP_{—}003318 (Homo sapiens); CAA18438 (Homo sapiens); 002765 (rabbit); P47741 (MOUSE); P43488 (MOUSE); P15725 (RAT); P23510 (HUMAN); AAC67236 (Rattus norvegicus); BAA20060 (Oryctolagus cuniculus); BAA 20059 (Oryctolagus cuniculus); AAB33944 (human); CAA53576 (Homo sapiens); CAA79772 (Mus musculus); CAA59476 (Mus musculus); AAA21871 (Mus musculus). Similarly, nucleic acids encoding OX40 with the following GenBank accession numbers can be used in the combination and combinatorial treatment method: AL022310 (human); AF037067 (Rattus norvegicus); AB003912 (rabbit); U12763 (Mus musculus).

In still another specific embodiment, 4-1BB protein with the following GenBank accession numbers can be used in the combination and combinatorial treatment method. 138427 (human); 138426 (human); 153384 (mouse); B32393 (mouse); P41273 (human); P41274 (mouse); Q07011 (human); P20334 (mouse); AAA93113 (mus musculus); AAA53134 (Homo sapiens); AAA53133 (Homo sapiens); AAA40167 (Mus musculus); AAA39435 (Mus musculus). Similarly, nucleic acids encoding 4-1BB with the following GenBank accession numbers can be used in the combination and combinatorial treatment method: AI664286; AII57872; AA109726; AA389045; AA155147; AA087107; W62906; U02567 (Mus musculus); U03398 (human); U03397 (human); J04492 (mouse); L15435 (Mus musculus).

Recently, a CD28/CTLA-4 homologue called ICOS has been cloned from activated human T cells. ICOS has an structure similar to CD28 and CTLA4 but does not have a conserved MYPPY motif, suggesting that it binds to unique ligand(s). Antibody cross-linking of ICOS enhances anti CD3-mediated T cell proliferation and cytokine production, although, unlike CD28, it does not enhance IL-2 production. ICOS protein and nucleic acid encoding ICOS protein with the following GenBank accession numbers can be used in the combination and combinatorial treatment method: S78540 (human) and AJ250559 (Mus musculus).

Also preferably, the costimulatory molecule is derived from an antigen presenting cell (APC), e.g., LFA-1, LFA-3, ICAM-1, ICAM-2, ICAM-3, CD 40 or B7.

In yet another aspect, the invention is directed to a combination, which combination comprises: a) an effective amount of an antagonist of NCAM L1; and b) an effective amount of another costimulatory inhibitory molecule. Preferably, the combination is in the form of a pharmaceutical composition. Additionally, the invention is directed to a method for reducing or inhibiting T cell activation, which method comprises administering an effective amount of an antagonist of NCAM L1 and an effective amount of another costimulatory inhibitory molecule to a mammal, wherein T cell reduction or inhibition is desirable, thereby reducing or inhibiting T cell activation in said mammal.

Any costimulatory inhibitory molecules can be used in the above combinations and methods. For example, the costimulatory inhibitory molecules can be antagonists of costimulatory molecules including the costimulatory molecules described above such as CD28, OX40, 4-1BB or ICOS and the costimulatory molecule is derived from an antigen presenting cell (APC), e.g., LFA-1, LFA-3, ICAM-1, ICAM-2, ICAM-3, CD 40 or B7.

In another example, the costimulatory inhibitory molecules used is T-lymphocyte-associated antigen 4 (CTLA-4) (Chambers and Allison, Curr. Opin. Cell. Biol., 11(2):203-10(1999)). CTLA-4 is an immunoglobulin supergene family glycoprotein that is expressed as homodimers on T cells. It binds to ligands B7.1 and B7.2 via the MYPPPY (in the single letter code for amino acids) motif in the immunoglobulin domain. CTLA-4 has a 10-fold higher affinity and a 100-fold higher avidity for B7 ligands compared to CD28 and exhibits distinct binding kinetics. The cytoplasmic tail of CTLA4 possess tyrosine-containing motifs postulated to be involved in signal transduction and protein trafficking. In another specific embodiment, CTLA-4 protein with the following GenBank accession numbers can be used in the combination and combinatorial treatment method: I46696 (rabbit); BAA08644 (oryctolagus cuniculus); P42081 (human); P42072; P16410; P09793; P33681; AAD50988 (Felis catus); AAD00698; AAD00697; (Rattus norvegicus); AD00696; (Mus musculus); 1AH; 2207257A; 1309302A; CAA63708 (Bos taurus); CAA29191 (Mus musculus); AAA86473 (Homo sapiens). Similarly, nucleic acids encoding CTLA-4 with the following GenBank accession numbers can be used in the combination and combinatorial treatment method: AF130428 (Marmota monax); D49844 (rabbit); AF143204 (canis familiaris breed beagle); AF1701725 (Felis catus); AF092740 (Ovis aries); AF153202 (Felix catus); U90271 (Rattus norvegicus); U37121 (Rattus norvegicus); L15006 (Homo sapiens); U17722 (Human).

The formulation, dosage and route of administration of the above-described compositions, combinations, preferably in the form of pharmaceutical compositions, can be determined according to the methods known in the art (see e.g., Remington: The Science and Practice of Pharmacy, Alfonso R. Gennaro (Editor) Mack Publishing Company, April 1997; Therapeutic Peptides and Proteins: Formulation, Processing, and Delivery Systems, Banga, 1999; and Pharmaceutical Formulation Development of Peptides and Proteins, Hovgaard and Frkjr (Ed.), Taylor & Francis, Inc., 2000; Medical Applications of Liposomes, Lasic and Papahadjopoulos (Ed.), Elsevier Science, 1998; Textbook of Gene Therapy, Jain, Hogrefe & Huber Publishers, 1998; Adenoviruses: Basic Biology to Gene Therapy, Vol. 15, Seth, Landes Bioscience, 1999; Biopharmaceutical Drug Design and Development, Wu-Pong and Rojanasakul (Ed.), Humana Press, 1999; Therapeutic Angiogenesis: From Basic Science to the Clinic, Vol. 28, Dole et al. (Ed.), Springer-Verlag New York, 1999). The compositions, combinations or pharmaceutical compositions can be formulated for oral, rectal, topical, inhalational buccal (e.g., sublingual), parenteral (e.g., subcutaneous, intramuscular, intradermal, or intravenous), transdermal administration or any other suitable route of administration. The most suitable route in any given case will depend on the nature and severity of the condition being treated and on the nature of the particular composition, combination or pharmaceutical composition which is being used.

The following example is included for illustrative purposes only and is not intended to limit the scope of the invention.

E. Examples

L1—Ligation is Required for Optimal Allo-Stimulation

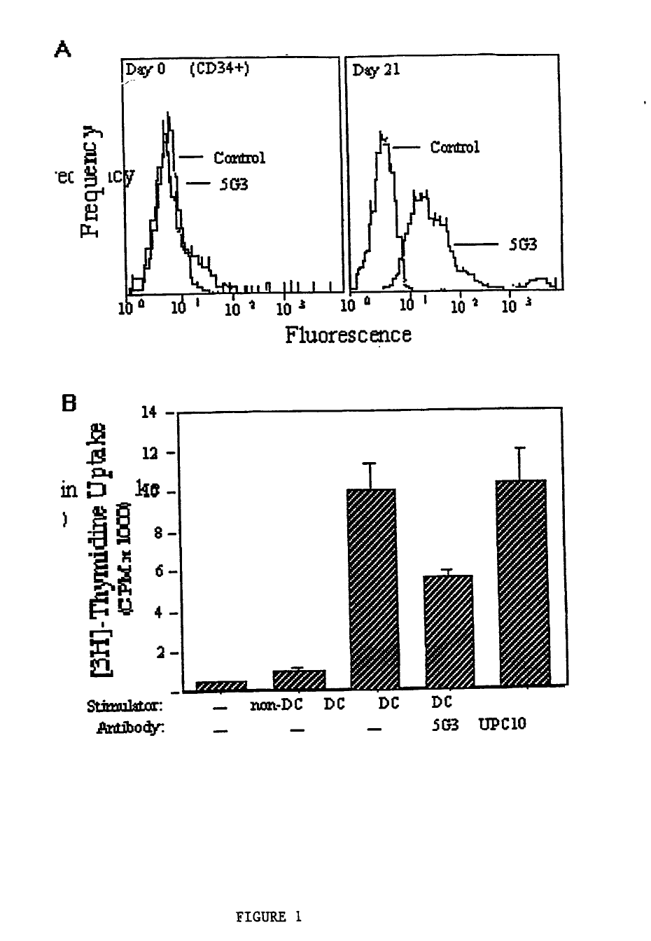

DCs are characterized as the most proficient APC in the immune system and are recognized to be the principal stimulators of primary MLR. A population of ‘stimulatory’ DC were produced from enriched CD34+ stem cells (>76% purity) using a combination of GM-CSF, SCF, IL-3, TNF-a, IL4. After 14-21 days these cells had acquired the expected DC morphology and phenotype (CD1a+, CD80+, CD86+, CD3−, CD14−, CD19−, CD56−) (data not shown). Importantly, acquisition of this DC phenotype was also marked by the induction of L1 expression (FIG. 1A). Expression of L1 on these DC is in accord with our previous report describing L1 expression on precursor and monocyte-derived DC (11). In order to establish an allogeneic MLR the L1+ DC were cocultured with PBMC from a different donor. The contribution of L1 to this allogeneic MLR was determined by the inclusion of a mAb specific for L1 (mAb 5G3; 11). Importantly, this antibody significantly reduced the allogeneic PBL response while an isotype-matched control antibody (UPC10) had no significant effect (FIG. 1B). Using PBMC from a number of different donors we observed levels of inhibition between 25-40% (not shown).

L1-Ligation Potentiates T-Cell Responses to PHA and to CD3

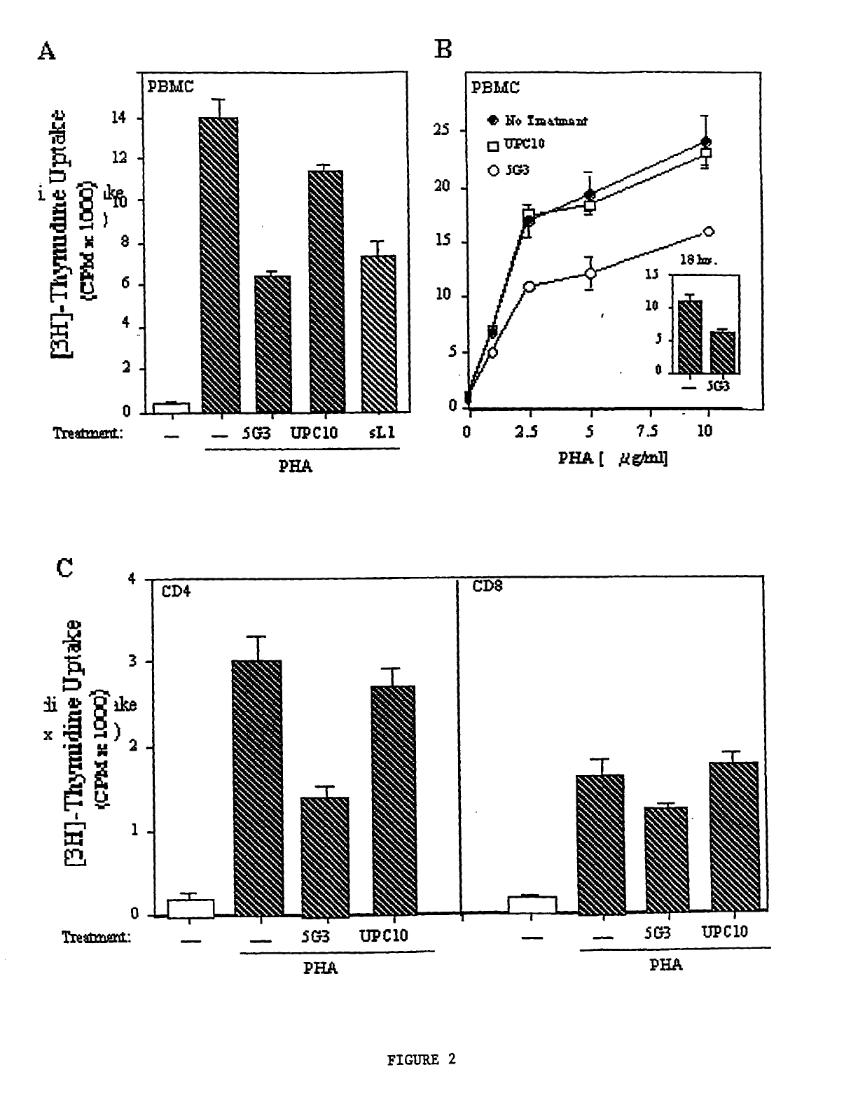

To further confirm costimulatory function in an autologous system we determined whether the anti L1 antibody 5G3 would also reduce proliferative responses to the T-cell mitogen PHA. Importantly, blockade of L1 by this mAb significantly reduced T-cell proliferation within the PBMC fraction (FIGS. 2A & B) and reduced the proliferation of enriched T-cell subsets, in particular CD4+ cells (FIG. 2C). Inhibition by antibody 5G3 was observed over a range of PHA concentrations (FIG. 2B) and as early as 18 hours after PHA stimulation (FIG. 2B inset).

Since we have previously demonstrated that mAb 5G3 will recognize monocytes in freshly isolated PBMC (11), it is likely that this antibody can inhibit mitogen-driven T-cell proliferation by blocking L1 expressed by these accessory cells. In this regard, either the removal of accessory cells or the blockade of costimulatory molecules expressed by these cells is known to abrogate or reduce T-cell responses to PHA. The importance of accessory cell function was confirmed in this study by the lower mitogenic responses of the isolated CD4+ and CD8+ T-cell subsets which were enriched to approximately 95% purity (FIG. 2). Finally, it is important to note that purified recombinant L1 (L1-ECD) was also found to reduce T-cell proliferation when offered as a soluble inhibitor (sL1: FIG. 2A).

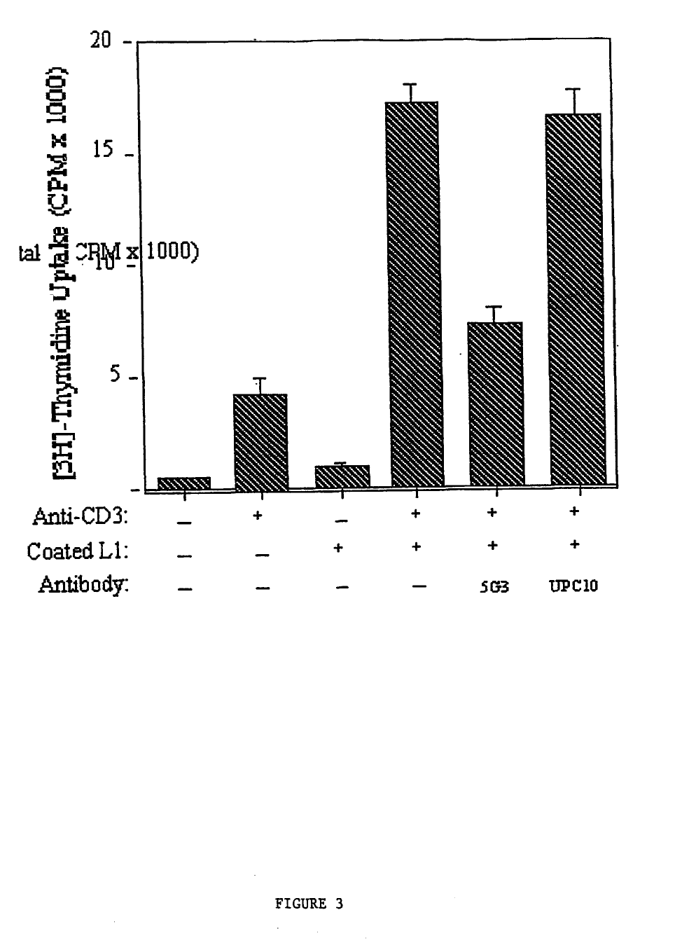

To further demonstrate costimulatory function it was determined whether purified immobilized L1 could potentiate polyclonal T-cell proliferation in response to ligation of the CD3 receptor (mAb OKT3). PBMC were added to wells precoated with mAb OKT3 alone or in combination with purified L1 ectodomain (L1-ECD). While wells coated with L1 alone failed to induce a significant response, the L1 did markedly enhance responses to the anti-CD3 mAb (FIG. 3). The specificity of this synergistic response was confirmed by inhibition with mAb 5G3 (FIG. 3). It is important to note that mAb 5G3 will block homophilic L1-mediated adhesion to purified L1 ECD but will not block integrin-dependent adhesion to the same L1-ECD preparation (not shown). This data confirms that L1 can function as a potent costimulatory molecule, and indicates that homophilic L1-L1 binding rather than direct L1-integrin binding is the stimulatory mechanism.

L1-Transfection of Myeloma Cells Promotes Adhesion via a Homophilic Mechanism and Potentiates MLR

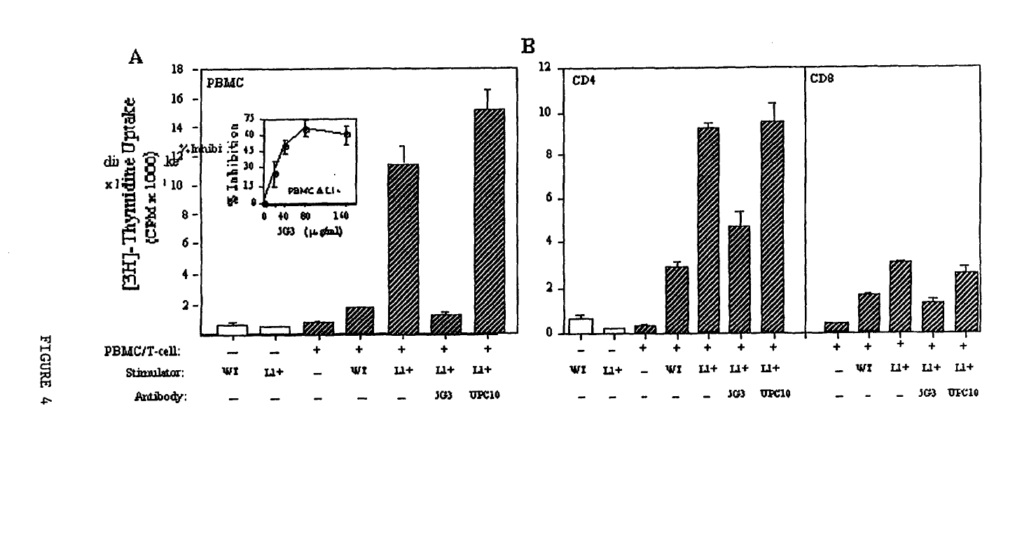

To directly demonstrate costimulatory function it should be possible to show that transfection and de novo expression of L1 will enhance the ability of the transfected cell to promote T-cell activation. We therefore compared the response of human PBMC or enriched T-cells to either wildtype or L1 transfected murine myeloma cells (J558L) in an in a one-way xenogeneic MLR. Importantly, irradiated myeloma cells manipulated to express high levels of human L1 (13), were found to be significantly more efficient at inducing PBMC proliferation than their wildtype counterparts (FIG. 4A). The specific contribution of L1 to this enhanced MLR was confirmed by inhibition with mAb 5G3 (FIG. 4A). Both CD4+ and CD8+ T-cells were found to respond differentially to the L1 transfected myeloma cells, however, the response of the CD4+ cells was found to be superior (FIG. 4B). The ability of the L1-transfected myeloma cells to induce T-cell proliferation was lost if these cells were cocultured with mononuclear cells derived from BALB/c mice (not shown). Since the J558L myeloma line was originally derived from a BALB/c mouse this would suggest that the ability of L1 to stimulate the proliferation of human T-cells is dependent upon a simultaneous recognition of; and response to, murine xenoantigens. In this regard, we did observe some T-cell proliferation in response to the wildtype L1-negative J558L cells (FIG. 4B).

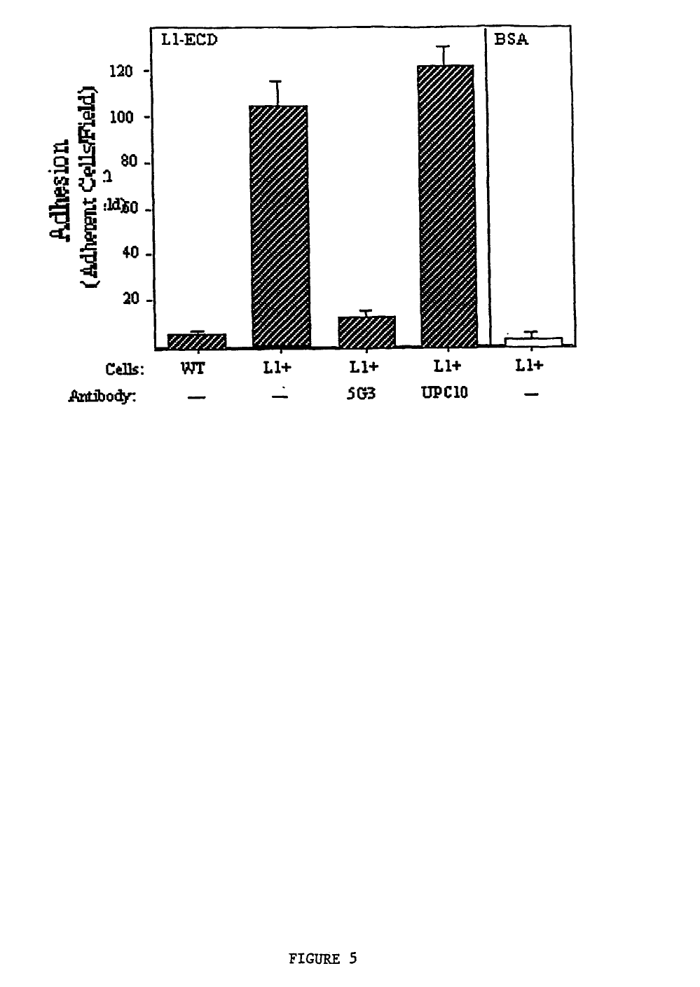

The introduction of L1 onto the surface of the myeloma cells was also found to facilitate adhesion to purified recombinant L1 (L1-ECD) via a homophilic mechanism (FIG. 5). These data suggest that the enhanced MLR observed with the L1-transfectants is due to a de novo capacity for L1-L1 interaction and adhesion. It is important to note, that the L1-mediated adhesion observed was completely abrogated by mAb 5G3 (FIG. 5) suggesting that this antibody can inhibit T-cell activation by virtue of its ability to prevent L1-L1 homophilic interaction.

It should be noted that inhibition of T-cell activation may depend upon the use of antagonists that can block L1-ligation without simultaneously causing L1 clustering and signalling. Supporting this concept we did not observe any significant inhibition of T-cell activation using an anti-L1 polyclonal antibody (data not shown). In this regard, it has been documented that polyclonal antibodies to L1 (unlike most mAbs and isolated soluble L1) can result in the activation of L1 dependent signalling pathways resulting the significant increases in intracellular cAMP levels (14). Finally, it was also observed that the inhibitory activity of 5G3 antibody preparations was critically dependent upon the removal of antibody aggregates by ultracentrifugation and that activity was lost both on freezing and concentration.

Concluding Remarks

Despite being described as a neural CAM we have recently documented L1 expression by ‘professional’ APC of both myelomonocytic and lymphoid origin, including B-cells, activated monocytes, monocyte derived DC, and follicular DC (11). L1 expression on monocyte-derived DC was induced after treatment with LPS (11) which is known to promote functional maturation or the acquisition of optimal costimulatory capacity. Based on these findings, and those present in this study, we propose that L1 expressed by such ‘professional’ APC can function as a costimulatory molecule in T-cell activation. A recent report documenting the expression of L1 by isolated peripheral blood T-cells (10) suggests a possible costimulatory mechanism based on homophilic L1-L1 ligation between the APC and T-cell. Adding support to this mechanism, we show that T-cell activation is inhibited by an anti-L1 mAb that also effectively prevents L1-L1 ligation.

Further detailed studies are required to define how L1-mediated signalling potentiates T-cell costimulation and to determine how L1 ranks along side other costimulatory molecules. A comparison with previous studies suggests that L1 is less important for T-cell co-stimulation than, for example, members of the B7 family. Thus inhibition of B7.1/B7.2 has been shown to reduce T cell proliferation to PHA and to allogeneic DC by up to 75 and 95% respectively (15, 16, 17). However, L1-mediated costimulation may be compared with other well documented co-stimulatory molecules such as CD58. Thus, blockade of CD2:CD58 binding has been shown to inhibit T-cell proliferation to PHA and to allogeneic DC by 30-35 and 45-50% respectively (16, 17).

While our findings indicate that homophilic L1-L1 ligation is required for T-cell costimulation, it is important to note that L1 can undergo multiple cis and trans interactions with other heterophilic ligands (6). For example, L1 has recently been shown to support a trans interaction with the integrin a5b1 (18); an integrin which has also been implicated in T-cell activation (19). It is conceivable that L1-integrin binding can contribute to costimulation after an initial T-cell activation event which is required for subsequent integrin activation and binding. In addition, it is notable that L1 can undergo cis-type interactions with both CD9 and CD24 both of which are known to play an important role in costimulation (20, 21).

Several important ramifications arise from the findings presented. Like other costimulatory molecules, L1 may contribute to the development of autoimmunity, graft rejection, and anti-tumor responses and in this context may prove to be a useful and novel target for immunotherapeutic intervention. The finding that soluble L1 can inhibit T-cell activation may prove significant given reports that aggressive neuroectodermal tumors can secrete large amounts of L1 (13, 22). Finally, high levels of L1-expression on post mitotic neurons and Schwann cells (2,6) suggest that this CAM may function as an important intermediary between nervous and immune system, particularly in the development of neuroimmunological disorders.

Materials and Methods

Reagents and Cell Lines

Anti-human L1 mAb 5G3 was generated in our laboratory (19). Purified recombinant L1 consisting of the entire extracellular domain of human L1 (L1-ECD) was kindly provided by Dr William Stallcup (The Burnham Institute, La Jolla, Calif.). J558L myeloma cells stably transfected with the full length human cDNA encoding for human L1 (J558L-L1; 21) were kindly provided by Dr Vance Lemmon (Case-Western Reserve University, Ohio.

Generation of DC and Enrichment of CD4 and CD8 T-Lymphocytes

C34+ cells were purified from normal cord blood using M-450 Dynabeads coated with an anti-CD34 mAb according to the manufacturers instructions. C34+-enriched (>76%) or CD34 negative cell populations were then cultured in the presence of granulocyte-macrophage colony-stimulating factor (10 ng/ml), human stem cell factor (40 ng/ml), human interleukin-3 (10 ng/ml), human tumor necrosis factor-a (100 U/mL) and human interleukin-4 (400 U/mL). After expansion for 7-21 days the levels of L1 expression on the cells was determined using anti-L1 mAb 5G3 directly conjugated to fluorescein isothiocyanate (FITC).

CD4+ and CD8+ cells were isolated from PBMC using M-450 Dynabeads coated with anti-CD4 or anti-CD8 mAbs. Isolation was according the manufacturers recommendations (Dynal, Fort Lee, N.J.). This method does not induce T-cell activation and resulted in approximately 95% purity.

MLR and Mitogen Assays

For mitogen assays, PBMC, or enriched CD4+, or CD8+ cells were cultured in 96-well round bottom plates (1×105 cells/well), with or without PHA (Sigma; 20 mg/ml). For ML assays, PBMCs (1×105 cells/well) or enriched CD4+, or CD8+ cells were co-cultured with irradiated wildtype or L1-transfected J558L cells at 1×104 cells/well or with irradiated cord blood derived dendritic cells (1×104 cells/well). Mitogen treated cells and cocultures were maintained for 3 days and the cultures pulsed with [3H]-thymidine (1 mCi/well) during the last 18 hours of the tree-day culture. The contribution of L1 to both mitogen and MLR assays was assessed by the incorporation of anti-L1 mAb 5G3 (80 mg/ml). Where appropriate an IgG2a isotype-matched control antibody (UPC10; 80 mg/ml) was also added.

Co-Stimulation of CD3-Mediated T-Cell Activation

Wells of a 96-well plate were pretreated with anti-CD3 antibody OKT3 (25 U/ml), with purified L1-ectodomain (40 mg/ml) or with a combination of both L1 and the antibody. After washing the wells, PBMCs were added to the precoated wells or to untreated wells for 72 hours. PBMC were added at 1×105 cells/well in the absence or presence of mAb 5G3, or in the presence of control antibody UPC10. Cultures were pulsed with [3H]-thymidine during the last 18 hours of a three day coculture. Treatments were performed in triplicate. Error bars are ±1SE.

Adhesion Assay

Purified L1-ECD fusion protein in PBS (30 mg/ml) was coated onto the bottom of 96-well Titertek plates essentially as described (13). The wells were then blocked with 5% BSA and wildtype or L1 transfected J558L myeloma cells added at 1×105/well and allowed to adhere for 60 minutes at 37° C. Cells were added in HBSS supplemented with 10 mM Hepes, BSA (0.5%) and CaCl2 (0.5 mM) (pH 7.4). For inhibition studies, the cells were pretreated with anti-L1 mAb 5G3 or isotype matched control mAb UPC10 at 80 mg/ml prior to the addition of both cells and inhibitors to pre-treated wells. Non-adherent cells removed under a constant vacuum and remaining adherent cells enumerated using a 40× objective as described (13).

### REFERENCES

1. Croft, M. and Dubey, C., Accessory molecule and costimulation requirements for CD4 T cell responses. Crit. Rev. Immunol. 1997. 17: 89-118.

2. Moos, M., Tacke R., Scherer, H., Teplow, D., Fruh K. and Schachner, M., Neural adhesion molecule L1 as a member of the immunoglobulin superfamily with binding domains similar to fibronectin. Nature 1988. 334: 701-703.

3. Cohen, N. R., Taylor, J. S. H., Scott, L. B., Guillery, R. W., Soriano, P. and Furley A. J., Errors in corticospinal axon guidance in mice lacking the neural cell adhesion molecule L1. Curr. Biol. 1997. 8: 26-33.

4. Dahme, M., Bartsch, U., Martini, R., Anliker, B., Schachner, M. and Mantei, N., Disruption of the gene coding for the cell adhesion molecule L1 leads to malformations of the nervous system in mice. Nat. Genet 1997. 17:346-349.

5. Lagenaur, C. and Lemmon, V., An L1-like molecule, the 8D9 antigen, is a potent substrate for neurite extension. Proc. Natl. Acad. Sci. USA 1987.84: 7753-7757.

6. Hortsch, M., The L1 family of neural cell adhesion molecules: old proteins performing new tricks. Neuron. 1996. 4: 587-593.

7. Felding-Habermann, B., Silletti, S., Mei F., Siu, C-H., Yip, P. M., Brooks, P. C., Cheresh, D.A., O'Toole, T. E., Ginsberg, M. H., and Montgomery, A. M. P., A single immunoglobulin-like domain of the human neural adhesion molecule L1 supports adhesion by multiple vascular and platelet integrins. J Cell Biol. 1997. 139: 1567-1581.

8. Kadmon, G., von Bohlen und Halbach, F., Horstkorte, R., Eckert, M., Altevogt P. and Schachner, M., Evidence for cis interaction and cooperative signalling by the heat-stable antigen nectadrin (murine CD24) and the cell adhesion molecule L1 in neurons. Euro. J. Neurosci. 1995. 7: 993-1004.

9. Schmidt, C., Kunemund, V., Wintergerst, E. S., Schmitz, B. and Schachner, M., CD9 of mouse brain is implicated in neurite outgrowth and cell migration in vitro and is associated with the a6b1 integrin and the neural adhesion molecule L1. J. Neurosci. Res. 1996. 43: 12-31.

10. Ebeling, O., Ducmza, A., Aigner, S., Geiger, C., Schollhammer, S. Kernshead, J. T., Moller, P., Schwartz-Albiez, R. and Altevogt, P., L1 adhesion molecule on human lymphocytes and monocytes: expression and involvement in binding to avb3 integrin. Eur. J. Immunuol. 1996. 26: 2508-2516.

11. Pancook, J. D., Reisfeld, R. A., Varki, N., Vitiello, A, Fox, R. I., and Montgomery A. M. P., Expression and regulation of the neural cell adhesion molecule L1 on human cells of myelomonocytic and lymphoid origin. J. Immunol. 1997. 158: 4413-4421.

12. Di Sciullo, G., Donahue, T., Schachner M. and Bogen, S. A., L1 antibodies block lymph node fibroblastic reticular matrix remodeling in vivo. J. Exp. Med. 1998. 187: 1953-1963.

13. Montgomery, A. M. P., Becker, J. C., Siu, C.-H., Lemmon, V. P., Cheresh, D. A., Pancook, J. D. Zhoa, X., and Reisfeld, R. A., Human neural cell adhesion molecule L1 and rat homologue NILE are ligands for integrin avb3. J. Cell Biol. 1996. 132: 475-485.

14. von Bohlen und Halbach, F., Taylor, J. and Schachner, J., Cell type-specific effects of the neural adhesion molecules L1 and N-CAM on diverse second messenger systems. Eur. J. Neurosci. 1992. 4: 896-903.

15. Nestle, F. O., Thompson, C., Shimizu, Y., Turka, L. A. And Nickoloff B. J., Costimulation of superantigen-activated T lymphocytes by autologous dandritic cells is dependent on B7. Cell. Immunol. 1994. 156: 220-229.

16. Woodward, J. E., Qin, L., Chavin, K. D., Lin J., Tono, T., Ding, Y., Linsley, P. S., Bromberg, J. S., and Baliga, P., Blockade of multiple costimulatory receptors induces hyporesponsiveness. Transplantation. 1996. 62: 1011-1018.

17. Young, J. W., Koulova, L., Soergel, S. A., Clark, E. A., Steinman, R. M., Dupont, B., The B7/BB1 antigen provides one of several costimulatory signals for the activation of CD4+ T lymphocytes by human blood dendritic cells in vitro. J. Clin. Invest. 90: 229-237.

18. Ruppert, M., Aigner, S., Hubbe, M., Yagita H. and Altevogt P., The L1 adhesion molecule is a cellular ligand for VLA-5. J. Cell Biol. 1995. 131: 1881-1891.

19. Maguire, J. E., Danahey, K. M., Burkly, L. C. and van Seventer, G. A., T cell receptor- and beta-1 integrin-mediated signals synergise to induce tyrosine phosphorylation of focal adhesion kinase pp125FAK) in human T cells. J. Exp. Med. 1995. 182: 2079-2090.

20. Liu, Y., Jones, B., Aruffo, A, Sullivan, K. M., Linsley, P. S. and Janeway C. A., Heat-stable antigen is a costimulatory molecule for CD4 T cell growth. J. Exp. Med. 1992. 175: 437-445.

21. Lagaudriere-Gesbert, C., Le Naour, F., Lebel-Binay, S., Billard, M., Lemichez, E., Boquet, P., Boucheix, C., Conjeaud, H., and Rubinstein. E., Functional analysis of four tetraspans, CD9, CD53, CD81, and CD82, suggests a common role in costimulation, cell adhesion, and migration: only CD9 upregulates HB-EGF activity. Cell Immunol. 1997. 182: 105-112.

22. Mujoo, K., Spiro, R. C., and Reisfeld. R. A., Characterization of a unique glycoprotein antigen expressed on the surface of human neuroblastoma Cells. J. Biol. Chem 1986. 261: 10299-10309.

Since modifications will be apparent to those of skill in this art, it is intended that this invention be limited only by the scope of the appended claims.

1. A method for potentiating T cell activation, which method comprises administering an effective amount of a multimerized neural cell adhesion molecule L1 (NCAM L1), or a functional derivative or fragment thereof, or a nucleic acid encoding said L1 or functional derivative or fragment thereof, or an agent that enhances production and/or costimulatory function of said L1 to a mammal wherein T cell activation is desirable, thereby potentiating T cell activation in said mammal.

2. The method of claim 1, wherein the NCAM L1, or a functional derivative or fragment thereof, is capable of L1-L1 homophilic interaction.

3. The method of claim 2, wherein the L1-L1 homophilic interaction mediates a L1-L1 ligation between an antigen presentation cell and a T cell or multimerization or crosslinking of L1 on a T cell.

4. The method of claim 1, wherein the NCAM L1, or a functional derivative or fragment thereof, directly or indirectly promotes an interaction with an integrin involved in T cell activation.

5. The method of claim 4, wherein the NCAM L1, or a functional derivative or fragment thereof directly or indirectly promotes a trans or cis interaction with the integrin α5β1 or αvβ3.

6. The method of claim 1, wherein the NCAM L1, or a functional derivative or fragment thereof, directly or indirectly promotes an interaction with a ligand involved in costimulation.

7. The method of claim 6, wherein the NCAM L1, or a functional derivative or fragment thereof, directly or indirectly promotes a cis-type interaction with CD9 and/or CD24.

8. The method of claim 1, wherein the agent enhances L1-L1 homophilic interaction between two NCAM L1, or a functional derivative or fragment thereof, or interaction between a NCAM L1, or a functional derivative or fragment thereof, and an integrin involved in T cell activation, or interaction between a NCAM L1, or a functional derivative or fragment thereof, and a ligand involved in costimulation.

9. The method of claim 1, wherein the mammal is a human and the NCAM L1, or a functional derivative or fragment thereof, is of human origin.

10. The method of claim 1, wherein the T cell to be activated is a CD4⁺ cell, a CD8⁺ cell or both.

11. The method of claim 1, wherein the mammal has tumor, cancer or infection.

12. The method of claim 1, wherein NCAM L1, or functional derivative or fragment thereof, or the nucleic acid encoding the NCAM L1, or functional derivative or fragment thereof, is delivered into an antigen presenting cell and the antigen presenting cell containing the NCAM L1 or the nucleic acid is then administered to the mammal.

13. A method for reducing or inhibiting T cell activation, which method comprises administering an effective amount of an antagonist of NCAM L1 to a mammal, wherein reduction or inhibition of T cell activation is desirable, thereby reducing or inhibiting T cell activation in said mammal.

14. The method of claim 13, wherein the antagonist of NCAM L1 is a protein, polypeptide or a peptide antagonist.

15. The method of claim 13, wherein the antagonist of NCAM L1 is a small molecule antagonist.

16. The method of claim 13, wherein the antagonist of NCAM L1 is selected from the group consisting of a NCAM L1 anti-sense oligonucleotide, an anti-NCAM L1 antibody, a soluble NCAM L1, or a derivative or fragment thereof, and an agent that reduces or inhibits production and/or costimulatory function of NCAM L1.

17. The method of claim 16, wherein the anti-NCAM L1 antibody is a monoclonal antibody.

18. The method of claim 17, wherein the anti-NCAM L1 monoclonal antibody is mAb 5G3.

19. The method of claim 13, wherein the antagonist of NCAM L1 reduces or inhibits L1-L1 homophilic interaction.

20. The method of claim 19, wherein the antagonist of NCAM L1 reduces or inhibits a L1-L1 ligation between an antigen presentation cell and a T cell.

21. The method of claim 20, wherein the antagonist of NCAM L1 reduces or inhibits a L1-L1 ligation without simultaneously causing NCAM L1 clustering and signaling.

22. The method of claim 13, wherein the antagonist of NCAM L1 reduces or inhibits NCAM L1's interaction with an integrin involved in T cell activation.

23. The method of claim 22, wherein the antagonist of NCAM L1 reduces or inhibits NCAM L1's trans or cis interaction with the integrin α5β1 or integrin αvβ3.

24. The method of claim 13, wherein the antagonist of NCAM L1 reduces or inhibits NCAM L1's interaction with a ligand involved in costimulation.

25. The method of claim 24, wherein the antagonist of NCAM L1 reduces or inhibits NCAM L1's interaction with CD9 and/or CD24.

26. The method of claim 13, wherein the mammal is a human.

27. The method of claim 13, wherein activation of a CD4⁺ cell, a CD8⁺ cell or both is reduced or inhibited.

28. The method of claim 13, wherein the mammal has a disease or disorder selected from the group consisting of autoimmunity, graft rejection and neuroimmunological disorders.

29. A combination, which combination comprises:
a) an effective amount of a multimerized neural cell adhesion molecule L1 (NCAM L1), or a functional derivative or fragment thereof, or a nucleic acid encoding said L1 or functional derivative or fragment thereof; or an agent that enhances production and/or costimulatory function of said L1; and
b) an effective amount of another costimulatory molecule, or an agonist thereof.

30. The combination of claim 29, which is in the form of a pharmaceutical composition.

31. The combination of claim 29, wherein the costimulatory molecule is selected from the group consisting of CD28, OX40, 4-1BB and ICOS.

32. The combination of claim 29, wherein the costimulatory molecule is derived from an antigen presenting cell (APC).

33. The combination of claim 32, wherein the APC-derived costimulatory molecule is selected from the group consisting of LFA-1, LFA-3, ICAM-1, ICAM-2, ICAM-3, CD 40 and B7.

34. A method for potentiating T cell activation, which method comprises administering an effective amount of a multimerized neural cell adhesion molecule L1 (NCAM L1), or a functional derivative or fragment thereof, or a nucleic acid encoding said L1 or functional derivative or fragment thereof, or an agent that enhances production and/or costimulatory function of said L1 and an effective amount of another costimulatory molecule to a mammal, wherein T cell activation is desirable, thereby potentiating T cell activation in said mammal.

35. A combination, which combination comprises:
a) an effective amount of an antagonist of NCAM L1; and
b) an effective amount of another costimulatory inhibitory molecule.

36. The combination of claim 35, which is in the form of a pharmaceutical composition.

37. The combination of claim 36, wherein the costimulatory inhibitory molecule is T-lymphocyte-associated antigen 4 (CTLA-4) or ethanol.

38. A method for reducing or inhibiting T cell activation, which method comprises administering an effective amount of an antagonist of NCAM L1 and an effective amount of another costimulatory inhibitory molecule to a mammal, wherein T cell reduction or inhibition is desirable, thereby reducing or inhibiting T cell activation in said mammal.
